(19) United States
(12) Patent Application Publication
(10) Pub. No.: US 2009/0264736 A1
(43) Pub. Date: Oct. 22, 2009

(54) DEVICE WITH 3D ARRAY OF STEERING COILS

(75) Inventors: Mark A. GRISWOLD, Shaker Heights, OH (US); Natalia Gudino, Shaker Heights, OH (US); Jeremiah A. Heilman, University Heights, OH (US)

Correspondence Address: KRAGULJAC & KALNAY, 4700 ROCKSIDE ROAD, SUMMIT ONE, SUITE 510, INDEPENDENCE, OH, 44131, US

(73) Assignee: CASE WESTERN RESERVE UNIVERSITY

(21) Appl. No.: 12/423,971

(22) Filed: Apr. 15, 2009

Related U.S. Application Data
(60) Provisional application No. 61/124,742, filed on Apr. 18, 2008.

(51) Int. Cl. A61B 5/055

(52) U.S. Cl. 600/423; 600/424

(57) ABSTRACT

Systems, methods, and other embodiments associated with remotely controlling a catheter configured with a 3D array of steering coils are described. One example magnetic resonance imaging (MRI) apparatus may include logic to remotely control a catheter. The 3D array of coils may include, for example, one axial coil and two side coils. The MRI apparatus may independently control current provided to members of the 3D array of coils. The MRI apparatus may also selectively produce different pulse sequences to mitigate and/or take advantage of signal voids present in an acquired image due to susceptibility effects from a field(s) generated by a member(s) of the 3D array of coils. Independently controlling the current provided to the 3D array of coils facilitates bending the catheter in a desired position as a result of a magnetic torque associated with a magnetic moment induced in a member of the 3D array of coils.

### PRIORITY CLAIM

This application claims the benefit of U.S. Provisional Application 61/124,742 filed Apr. 18, 2008, titled Catheter With 3D Array of Steering Coils, by the same inventors.

### COPYRIGHT NOTICE

A portion of the disclosure of this patent document contains material that is subject to copyright protection. The copyright owner has no objection to the facsimile reproduction of the patent document or the patent disclosure as it appears in the Patent and Trademark Office patent file or records, but otherwise reserves all copyright rights whatsoever.

### BACKGROUND

Endovascular procedures involve moving a catheter tip through a blood vessel(s). It may be challenging to direct a catheter tip into a small vessel. It may also be challenging to navigate a catheter tip through a complex set of blood vessels. Some conventional approaches to guide a catheter tip may have been based on x-rays. However, x-ray angiography may be limited to projection views and may pose risks associated with radiation exposure. Other conventional approaches to guide a catheter tip may have involved interventional magnetic resonance imaging (IMRI).

IMRI facilitates catheter guidance in a three dimensional space without using ionizing radiation. Conventional IMRI guidance systems may have relied on a single axial coil to reshape (e.g., bend) a catheter in a single direction. The axial coil response may have depended on an initial angle between the main magnetic field and the catheter. The deflection attainable using the axial coil may have been limited. Additionally, signal voids associated with the coil may have compromised image quality.

Magnetic resonance imaging (MRI) is an alternative to digital subtraction angiography (DSA) for image-guidance of endovascular procedures. Real-time MRI facilitates visualizing soft tissue anatomy that may not be possible with projection X-ray images. The magnetic field in an MR scanner is a relatively unique environment. In this environment, a single dimensional endovascular catheter steering mechanism has been described. Roberts, et al. (cited below) demonstrated that current applied to a solenoid located at the tip of a catheter can bend the catheter when the catheter is in the unique environment of the MRI scanner magnetic field. Since the catheter can be bent in a single direction, rudimentary steering is possible. The catheter can be bent as a result of a magnetic moment induced in the solenoid in the catheter. See, for example, Roberts T P, Hassenzahl W V, Hetts S W, Arenson R L, Remote Control of Catheter Tip Deflection: An Opportunity for Interventional MRI, Magn Reson Med 2002; 48(6):1091-5.

### BRIEF DESCRIPTION OF THE DRAWINGS

The accompanying drawings, which are incorporated in and constitute a part of the specification, illustrate various example systems, methods, and other example embodiments of various aspects of the invention. It will be appreciated that the illustrated element boundaries (e.g., boxes, groups of boxes, or other shapes) in the figures represent one example of the boundaries. One of ordinary skill in the art will appreciate that in some examples one element may be designed as multiple elements or that multiple elements may be designed as one element. In some examples, an element shown as an internal component of another element may be implemented as an external component and vice versa. Furthermore, elements may not be drawn to scale.

FIG. 1 illustrates an example method associated with intravascular catheter control using a 3D array of active steering coils.

FIG. 2 illustrates an example method associated with intravascular catheter control using a 3D array of active steering coils.

FIG. 3 illustrates components associated with an example catheter having a three dimensional (3D) array of steering coils.

FIG. 4 illustrates components associated with an example catheter operably connected to a control logic that is to individually control members of the 3D array of steering coils.

FIG. 5 illustrates an MRI apparatus associated with intravascular catheter control using a 3D array of active steering coils.

FIG. 6 illustrates a device configured with three elements arranged as a 3D array of steering elements.

FIG. 7 illustrates the device of FIG. 6 with the three elements separated from the device to facilitate understanding how a magnetic torque is generated by individually controlling current flowing in an element.

FIG. 8 illustrates another device configured with a different arrangement of coils.

FIG. 9 illustrates forces associated with single coil catheter tip deflection as described by University of Toronto Student F Settecase in the Master's Thesis titled: Magnetically-Assisted Remote Control (MARC) Steering of Endovascular Catheters for Interventional MRI: A Model For Deflection and Design Implications, as reported in Med Phys. 2007 August; 34(8):3135-42. This work cites the previous single coil catheter deflection work performed by Roberts, et al., cited above in the background section.

FIG. 10 illustrates a catheter with a joint, and coils associated with producing a relative motion between portions of the catheter on different sides of the joint.

### DETAILED DESCRIPTION

Example apparatus include multiple elements that can be individually controlled to facilitate reshaping a device located in a constant magnetic field. The individual elements may be coils, the device may be a catheter, and the constant magnetic field may be produced by an MRI scanner. Example systems and methods concern controlling a device (e.g., catheter) that may be selectively manipulated (e.g., bent) in multiple directions in multiple (e.g., three) axes as a function of interactions between the magnetic field present in an MR scanner and a magnetic moment created when a current is applied to a coil(s) in the device.

The coils may be arranged in a three dimensional array. A catheter configured with the three dimensional array of coils may be able to be navigated and thus may be suitable for use in endovascular procedures. An example catheter is configured with the three dimensional (3D) array of coils rather than only an axial coil(s) as in conventional systems. The 3D array of coils may include multiple (e.g., three) individually controllable coils that may each be oriented in different directions. As the catheter is moved through the vasculature, the catheter may be bent in different directions by selectively creating a magnetic moment(s) by applying a current(s) to a coil(s). In a conventional system, a catheter arranged with a single axial coil might only be bent or deflected in one direction.

A device (e.g., catheter) configured with a 3D array of coils facilitates generating both stronger and more complex deflections than single axial coil only systems. With stronger and more complex deflections available, example systems and methods facilitate performing enhanced visualization techniques for a device being tracked, for an area surrounding the device, and combinations thereof. The stronger and more complex deflections can produce stronger and more complex magnetic moments to create magnetic torque. However, the magnetic moment(s) may also cause local magnetic field inhomogeneity in the region of the induced magnetic moment(s). Because the local magnetic field is inhomogeneous while the magnetic moment is present, signal from proton spins in that local region may be lost. The lost signal may provide a signal void in an MR image. Since the coil(s) that produced the signal void is located in the area of the signal void, the signal void can be used to passively track the coil(s). The void will be present while current to the coil(s) is on, but will not be present while current to the coil(s) is off.

One example system includes a micro-catheter configured with three coils. The three coils may be manufactured into the catheter tip, inserted into the catheter tip, and so on. The microcatheter may be, for example, a 2.5 FR microcatheter. The three coils may be constructed from, for example, 42 gauge wire. While a 2.5 FR microcatheter and 42 gauge wire are described, it is to be appreciated that other sized microcatheters and other gauges of wire may be employed.

The three coils may be arranged as a 3D array of steering coils. A “3D array of steering coils” refers to a set of elements in which a magnetic moment can be produced in any spatial direction by applying a set of currents to the elements when the elements are located in a magnetic field. The magnetic moment creates a magnetic torque that can deflect an apparatus (e.g., catheter) housing the 3D array of steering coils. Since the apparatus can be bent, it can be “steered” during an endovascular procedure. In one example, a collection of coils may include two 70 turn axial coils and a 15 turn square side coil. In this configuration the axial coils may be separated by 1 centimeter and the side coil may be 2×4 mm². While 70 turn and 15 turn coils are described, it is to be appreciated that other turn counts may be employed. Similarly, while 1 cm spacing and 2×4 mm² sizing are described, it is to be appreciated that other spacings and sizes may be employed. In one example, at least two coils in the array of coils are to be arranged along the length of the catheter.

In one example, individual members of the 3D array of coils may be independently controlled with respect to current applied to the members. Thus, individual magnetic moments may be individually controllable. The control may be executed, for example, by a control circuit, by a control logic, by a specially programmed microprocessor, by a computer, and so on. Individual current control facilitates both coarse (e.g., on/off) control and fine-grained (e.g., variable current) control of members of the 3D array of coils.

A catheter arranged with a 3D array of coils may be used in conjunction with acquiring image data using a clinical MRI scanner. The MRI scanner may be configured to interact with (e.g., remotely control) a catheter configured with a 3D array of active steering coils. The remote control may include, for example, turning current on/off for a coil, turning current on/off for a set of coils, varying the current in a coil, varying the current in a set of coils, and so on.

A catheter may have an initial position when inserted into a subject. This position may be described by measurements including an initial inclination angle. Example systems and methods may measure deflection angles with respect to the initial inclination angle. Since a catheter may have multiple elements (e.g., coils) that can cause deflections, multiple inclination angles and deflection angles may be measured. The deflection angles may facilitate locating the catheter, deciding how to relocate the catheter, relocating the catheter, deciding what current(s) to apply to what coil(s), applying a current(s) to a coil(s), and so on. The deflection angles may be measured using, for example, a real-time Fast Low Angle Shot (FLASH) sequence.

Conventional systems required establishing a pre-defined relationship between the single axial coil catheter initial position and the MRI scanner main magnetic field. Example systems and methods are not so limited. The additional coils in an example 3D array of coils facilitate having the catheter function regardless of the initial angle with respect to B₀ (the main magnetic field produced by an MR apparatus).

Example systems and methods may employ different imaging approaches to acquire different images. One imaging approach may be targeted at acquiring images of tissues surrounding the catheter. The tissues may be, for example, blood vessel walls, organ walls, and so on. In one example, current to a catheter coil(s) may be manipulated to mitigate issues associated with susceptibility voids that are present when a member(s) of the 3D array of coils is active. For example, current to a catheter coil(s) may be switched off during a portion(s) of a pulse sequence. This may facilitate visualizing surrounding tissue without interference from the catheter. In one example, the pulse sequence may be a FLASH sequence with parameters of 25 degree flip angle, 200 mm field of view (FOV), and 10 mm slice thickness. Other pulse sequence parameters (e.g., timings) may be set to mitigate issues associated with catheter motion. These parameter settings may balance issues associated with, for example, attaining a short readout window to maintain catheter position in an acquired image. In one example, catheter motion may be acceptably small when a readout window is less than 2 ms. In one example, deflection may be improved and/or optimized when TR is greater than 15 ms.

Another imaging approach may be targeted at visualizing the catheter itself. In this example, a pulse sequence that is different from the pulse sequence used to visualize surrounding tissues may be employed. For example, a FLASH sequence without slice select rewinding gradients may be employed. The pulse sequence, in combination with current in the coils, may produce a void in nuclear magnetic resonance (NMR) signal produced by the item. Since the coils are in the void, the location of the void can serve as a proxy for the location of the coils and thus the device.

The following includes definitions of selected terms employed herein. The definitions include various examples and/or forms of components that fall within the scope of a term and that may be used for implementation. The examples are not intended to be limiting. Both singular and plural forms of terms may be within the definitions.

References to “one embodiment”, “an embodiment”, “one example”, “an example”, and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

“Computer-readable medium”, as used herein, refers to a medium that stores signals, instructions and/or data. A computer-readable medium may take forms, including, but not limited to, non-volatile media, and volatile media. Non-volatile media may include, for example, optical disks, magnetic disks, and so on. Volatile media may include, for example, semiconductor memories, dynamic memory, and so on. Common forms of a computer-readable medium may include, but are not limited to, a floppy disk, a flexible disk, a hard disk, a magnetic tape, other magnetic medium, an ASIC, a CD, other optical medium, a RAM, a ROM, a memory chip or card, a memory stick, and other media from which a computer, a processor or other electronic device can read.

“Logic”, as used herein, includes but is not limited to hardware, firmware, software in execution on a machine, and/or combinations of each to perform a function(s) or an action(s), and/or to cause a function or action from another logic, method, and/or system. Logic may include a software controlled microprocessor, a discrete logic (e.g., ASIC), an analog circuit, a digital circuit, a programmed logic device, a memory device containing instructions, and so on. Logic may include one or more gates, combinations of gates, or other circuit components. Where multiple logical logics are described, it may be possible to incorporate the multiple logical logics into one physical logic. Similarly, where a single logical logic is described, it may be possible to distribute that single logical logic between multiple physical logics.

An “operable connection”, or a connection by which entities are “operably connected”, is one in which signals, physical communications, and/or logical communications may be sent and/or received. An operable connection may include a physical interface, an electrical interface, and/or a data interface. An operable connection may include differing combinations of interfaces and/or connections sufficient to allow operable control. For example, two entities can be operably connected to communicate signals to each other directly or through one or more intermediate entities (e.g., processor, operating system, logic, software). Logical and/or physical communication channels can be used to create an operable connection.

“Signal”, as used herein, includes but is not limited to, electrical signals, optical signals, analog signals, digital signals, data, computer instructions, processor instructions, messages, a bit, a bit stream, or other means that can be received, transmitted and/or detected.

“User”, as used herein, includes but is not limited to one or more persons, software, computers or other devices, or combinations of these.

Some portions of the detailed descriptions that follow are presented in terms of algorithms and symbolic representations of operations on data bits within a memory. These algorithmic descriptions and representations are used by those skilled in the art to convey the substance of their work to others. An algorithm, here and generally, is conceived to be a sequence of operations that produce a result. The operations may include physical manipulations of physical quantities. Usually, though not necessarily, the physical quantities take the form of electrical or magnetic signals capable of being stored, transferred, combined, compared, and otherwise manipulated in a logic, and so on. The physical manipulations create a concrete, tangible, useful, real-world result.

It has proven convenient at times, principally for reasons of common usage, to refer to these signals as bits, values, elements, symbols, characters, terms, numbers, and so on. It should be borne in mind, however, that these and similar terms are to be associated with the appropriate physical quantities and are merely convenient labels applied to these quantities. Unless specifically stated otherwise, it is appreciated that throughout the description, terms including processing, computing, determining, and so on, refer to actions and processes of a computer system, logic, processor, or similar electronic device that manipulates and transforms data represented as physical (electronic) quantities.

Example methods may be better appreciated with reference to flow diagrams. While for purposes of simplicity of explanation, the illustrated methodologies are shown and described as a series of blocks, it is to be appreciated that the methodologies are not limited by the order of the blocks, as some blocks can occur in different orders and/or concurrently with other blocks from that shown and described. Moreover, less than all the illustrated blocks may be required to implement an example methodology. Blocks may be combined or separated into multiple components. Furthermore, additional and/or alternative methodologies can employ additional, not illustrated blocks.

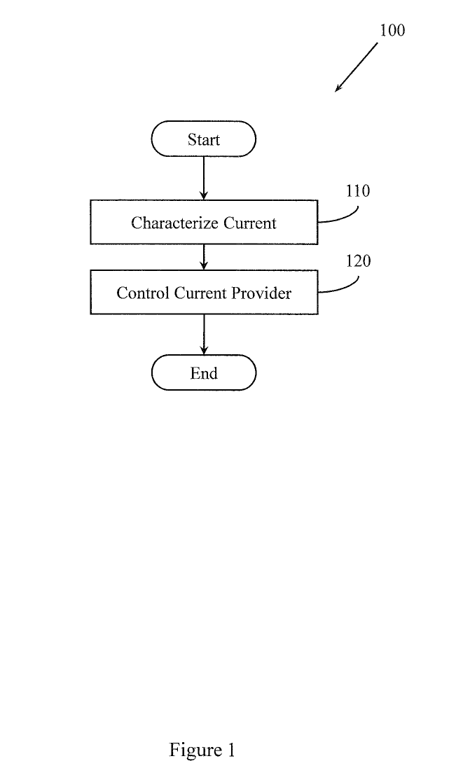

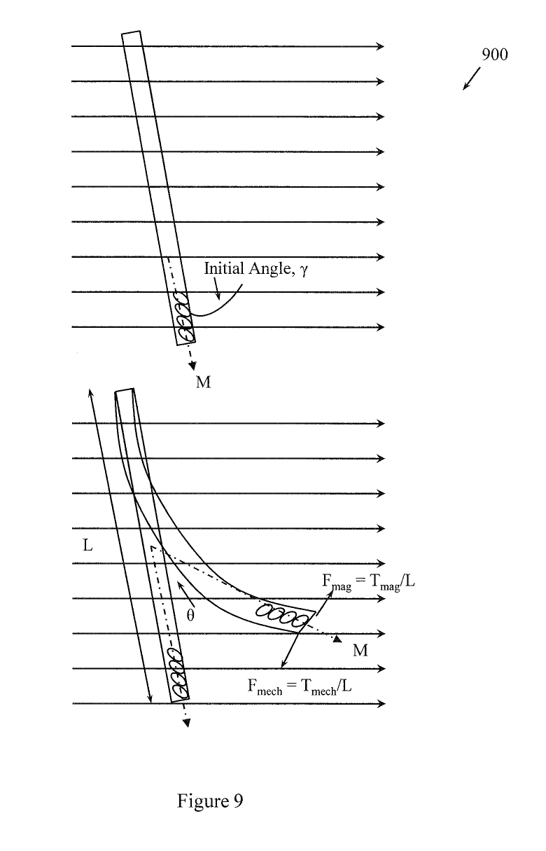

FIG. 1 illustrates a method 100 associated with remotely controlling a catheter configured with a 3D array of active steering coils. Method 100 includes, at 110, determining one or more of, a current amount, and a current polarity for a current to be provided to a member of a three dimensional (3D) array of steering coils coupled to a catheter. The current will be provided while the catheter is positioned in a magnetic field (e.g., B₀) produced by an MRI apparatus. In one example, the current amount and the current polarity are configured to produce a magnetic torque sufficient to bend the catheter in a desired direction and in a desired amount. Recall that the magnetic torque is a result of a magnetic moment induced in the at least one member of the 3D array of steering coils as a function of an interaction between the magnetic field produced by the MRI apparatus and the magnetic moment. FIG. 9 illustrates the forces associated with bending a catheter in a single direction using magnetic torque.

With the current amount and the current polarity determined, method 100 also includes, at 120, controlling a current provider to provide the current to the at least one member of the 3D array of steering coils. The current will be provided with the determined current amount and current polarity. Providing the current while the catheter is positioned in the magnetic field will produce the magnetic moment that will in turn produce the magnetic torque that will cause the catheter to bend in the desired amount in the desired direction. Bending the catheter in the desired direction and the desired amount facilitates steering the catheter through the vasculature. Thus, in one example, the current amount and the current polarity are determined as a function of a desired navigational movement of the catheter during an endovascular procedure.

The current amount, current polarity, and even which coil is to be provided current may depend on an orientation of the catheter and coils with respect to the direction of the main magnetic field. Thus, in one example, method 100 may include measuring a set of deflection angles associated with an inclination angle of the catheter with respect to the magnetic field produced by the MRI apparatus. Once the deflection angles are known, method 100 may include identifying a selected member of the 3D array of steering coils to be provided with a current based, at least in part, on a subset of the set of deflection angles. Having identified the coil to be provided with current, method 100 may include identifying the current amount and the current polarity for the current to be provided to the selected member of a 3D array of steering coils based, at least in part, on a subset of the set of deflection angles.

While FIG. 1 illustrates various actions occurring in serial, it is to be appreciated that various actions illustrated in FIG. 1 could occur substantially in parallel. By way of illustration, a first process could characterize current and a second process could control a current provider. While two processes are described, it is to be appreciated that a greater and/or lesser number of processes could be employed and that lightweight processes, regular processes, threads, and other approaches could be employed. In one example, computer-executable instructions for performing method 100 may be stored on a computer-readable medium.

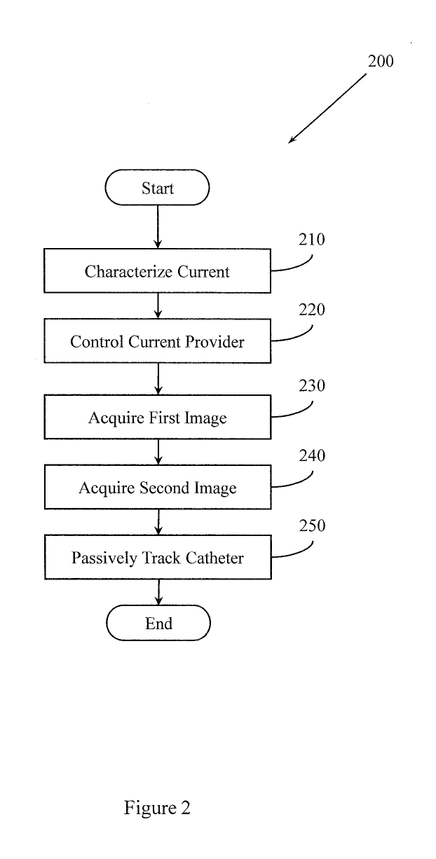

FIG. 2 illustrates a method 200 associated with remotely controlling a catheter configured with a 3D array of coils. Method 200 includes actions similar to those described in connection with method 100. For example, method 200 includes, at 210, characterizing the current and, at 220, controlling a current provider. However, method 200 includes additional actions.

For example, method 200 may also include, at 230, controlling the MRI apparatus to acquire a first image in the region of the catheter while the current provider is providing current to at least one member of the 3D array of steering coils. This first image may include a signal void associated with the magnetic moment. The magnetic moment may disrupt the homogeneity of the main magnetic field B₀ and thus may impact the susceptibility of spins to a specific energizing frequency. Thus, certain spins in the region of the coils producing the magnetic moment may not contribute to acquired NMR signal. This signal void may be useful as a proxy for the location of the coil producing the magnetic moment.

Therefore, method 200 may also include, at 240, controlling the MRI apparatus to acquire a second image in the region of the catheter while the current provider is not providing current to any member of the 3D array of steering coils. With the two images available, one having a signal void and one not having a signal void, method 200 may include, at 250, passively tracking the catheter based, at least in part, on the first image and the second image.

In one example, the images acquired at 230 and 240 may be acquired using a FLASH pulse sequence. In one example, at least one of the images acquired at 230 and 240 may be acquired using a pulse sequence that does not include slice select rewinding gradients.

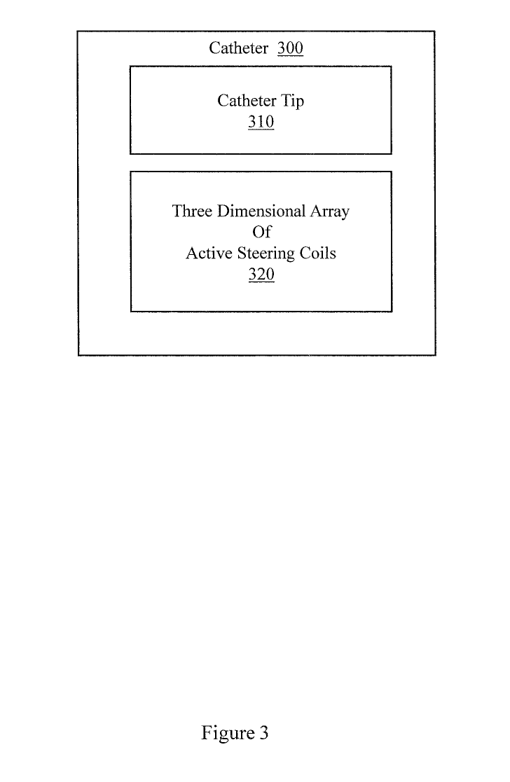

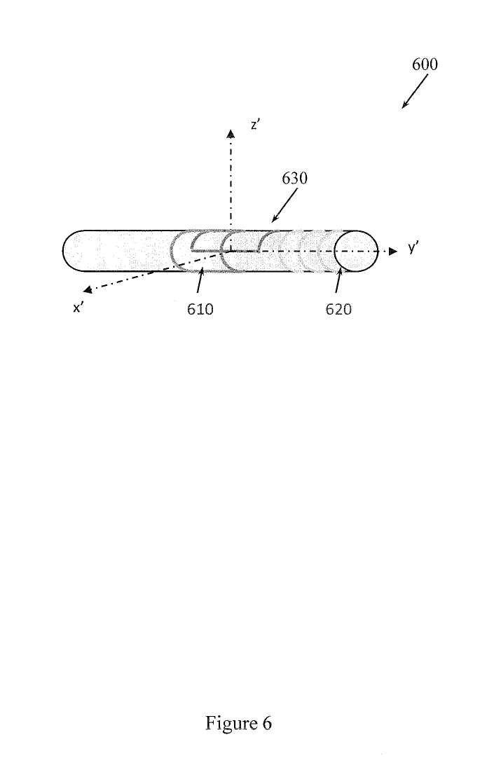

FIG. 3 illustrates elements of a catheter 300. One skilled in the art will recognize that FIG. 3 represents catheter concepts without representing an actual catheter. Catheter 300 includes a catheter tip 310 and a 3D array of active steering coils 320. In one example the catheter 300 is a microcatheter. In one example, the 3D array of active coils 320 includes at least three coils. The three coils may include, for example, two side coils and an axial coil. The array of coils 320 may be manufactured into the tip 310, may be inserted into the tip, and so on. At least two of the coils may be arranged along the length of the catheter. While FIG. 3 represents a conceptual catheter, FIG. 6 illustrates an actual example catheter 600.

Catheter 600 includes three separate coils oriented in three different directions to facilitate bending catheter 600 in different directions. A first coil 610 is oriented to be able to produce a magnetic moment in the x direction. A second coil 620 is oriented to be able to produce a magnetic moment in the y direction and a third coil 630 is oriented to be able to produce a magnetic moment in the z direction. While three coils arranged to produce magnetic moments in three orthogonal and intersecting axes are illustrated, one skilled in the art will appreciate that a different number of coils may be arranged in different locations to facilitate bending a device (e.g., catheter) in different manners.

Thus, more generally, FIG. 6 illustrates an apparatus. The apparatus includes a flexible housing (e.g., catheter 600) configured to be bendable in three or more directions when located in a magnetic field. The apparatus also includes a three dimensional array of elements (e.g., coil 610, coil 620, coil 630) coupled to the flexible housing. Members of the three dimensional array of elements are configured to be controlled individually with respect to current applied to the members. Members of the three dimensional array of elements are configured to produce a magnetic torque sufficient to bend the flexible housing when supplied with a current while the apparatus is positioned in the magnetic field.

In one example, the flexible housing is a catheter configured for use in an endovascular procedure and the magnetic field is generated by an MRI apparatus configured to acquire an image of the catheter during the endovascular procedure. In this example, the three dimensional array of elements is a three dimensional array of coils. Recall that being “three dimensional” refers to the ability to reshape the catheter in three dimensions.

Different catheters may be configured with different arrays of coils. In one example, the three dimensional array of elements is a three dimensional array of coils that includes at least two axial coils. In one example, the three dimensional array of elements includes at least one side coil. The side coil may be, for example, a square side coil. In one example, the three dimensional array of elements includes two side coils and an axial coil.

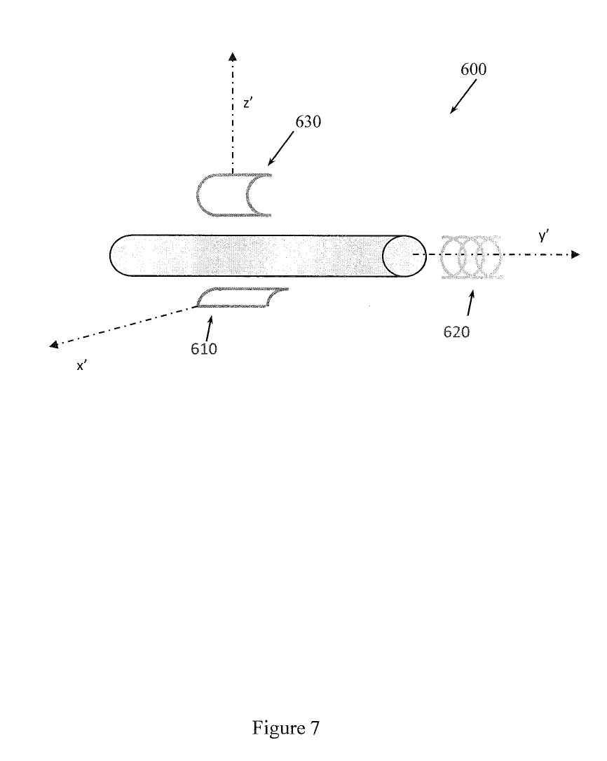

FIG. 7 illustrates the coils associated with catheter 600 separated from catheter 600 to facilitate understanding the positioning of the coils and the deflections that can be produced when current is supplied to the different coils.

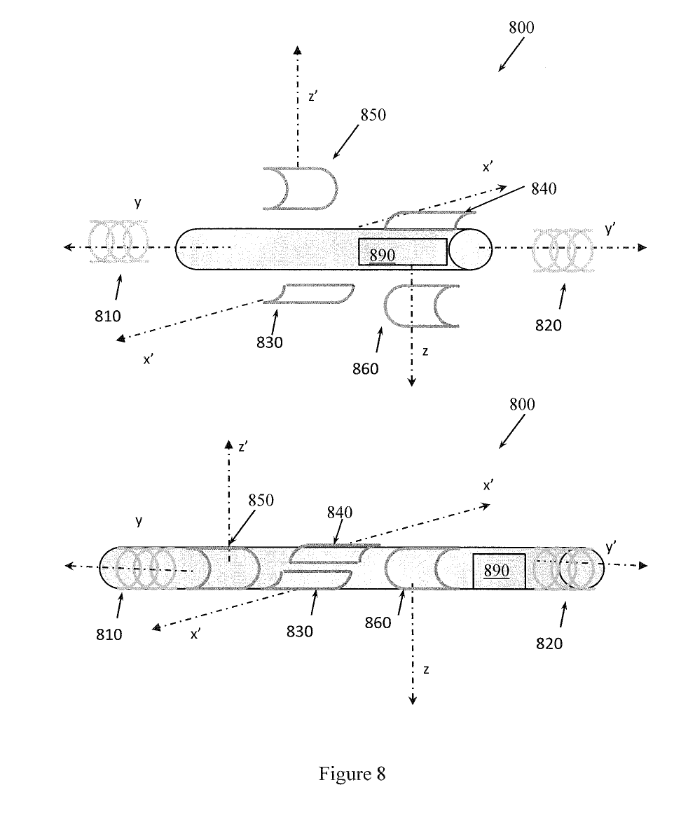

FIG. 8 illustrates another catheter 800 having a different number and configuration of coils. Catheter 800 is configured with two coils 810 and 820 that are configured to produce magnetic moments in the y direction. Similarly, catheter 800 is configured with a pair of coils 830 and 840 that are configured to produce a magnetic moment in the x direction and a pair of coils 850 and 860 that are configured to produce a magnetic moment in the z direction. While six coils arranged in three pairs of two are illustrated, one skilled in the art will appreciate that other configurations are possible. Pairs of coils may facilitate producing a desired deflection without having to control the polarity of a current.

FIG. 8 also illustrates catheter 800 configured with a “payload” apparatus 890. Recall that catheter 800 may be involved in an endovascular procedure. Once the catheter 800 has been steered to an interesting location using the 3D array of steering coils, it may be desirable to perform an action. Therefore, in one example, payload apparatus 890 may be a fiber optic element coupled to the catheter 800. The fiber optic element may be configured to provide a fiber optic image of an item in the region of the catheter 800. In another example, the payload apparatus 890 may be a laser ablation element coupled to the catheter 800. The laser ablation element may be configured to ablate an item in the region of the catheter 800.

Apparatus 300, apparatus 600, and apparatus 800 may also include a current source. The current source may be coupled to the housing and to the three dimensional array of elements. The current source may be configured to provide current to members of the three dimensional array of elements. In one example, the current source is a conductor configured to be connected to an external current provider. In another example, the current source may be configured to have a current induced in the current source as the result of being exposed to radio-frequency (RF) energy.

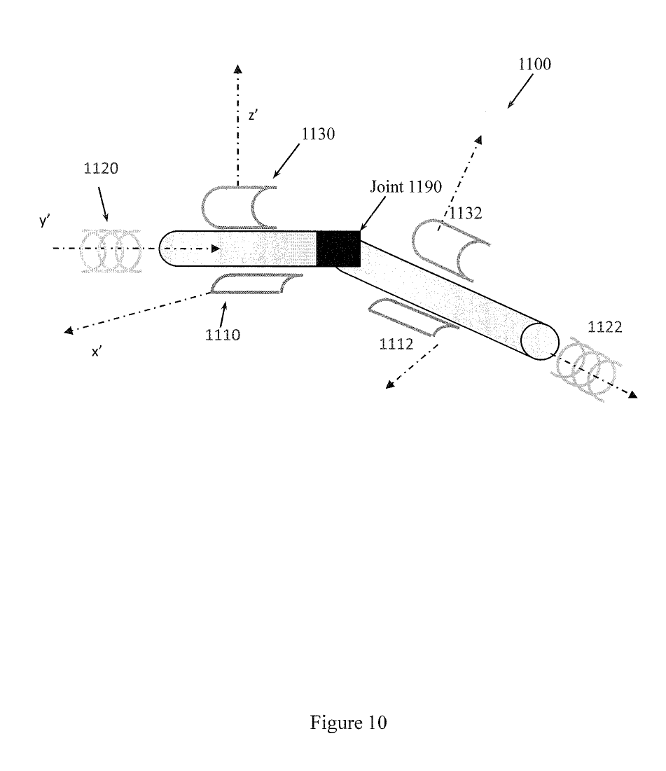

FIG. 10 illustrates a catheter 1100 having a joint 1190. Catheter 1100 also includes coils arranged and configured to facilitate producing a relative motion around joint 1190. Catheter 1100 includes a first catheter portion and a second catheter portion coupled by joint 1190. Catheter 1100 also includes a set of coils configured to produce a set of magnetic torques when provided with a current while positioned in a magnetic field produced by an MRI apparatus. The set of magnetic torques is configurable to produce a relative motion between the first catheter portion and the second catheter portion about the joint 1190. FIG. 10 illustrates six coils arranged in three pairs of two (e.g., coils 1110, 1112, coils 1120, 1122, and coils 1130, 1132).

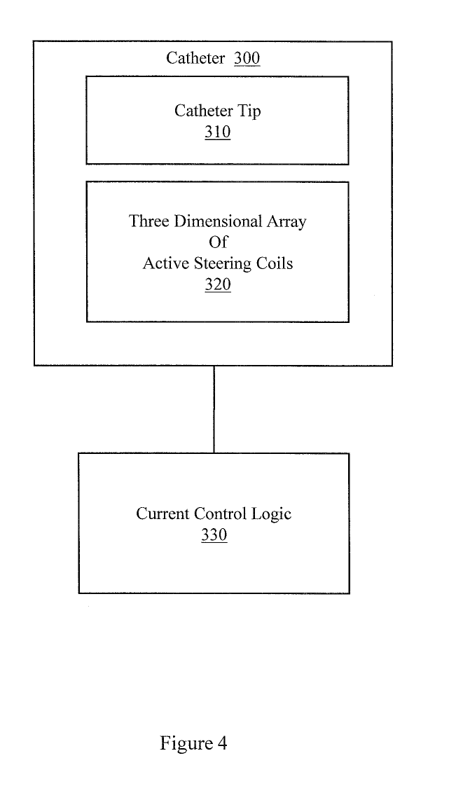

FIG. 4 illustrates catheter 300 where individual members of the 3D array of active coils 320 are independently controllable with respect to current. The current provided to the members of the 3D array may be controlled by a current control logic 330. While a current control logic 330 is illustrated, it is to be appreciated that current may be controlled by a current control logic, a current control circuit, a microprocessor programmed to control current, and so on. The individual control illustrated in FIG. 4 may be applied to coils associated with catheters illustrated in FIGS. 6, 7, 8, and 10. The current control logic 330 may be a portion of an MRI apparatus.

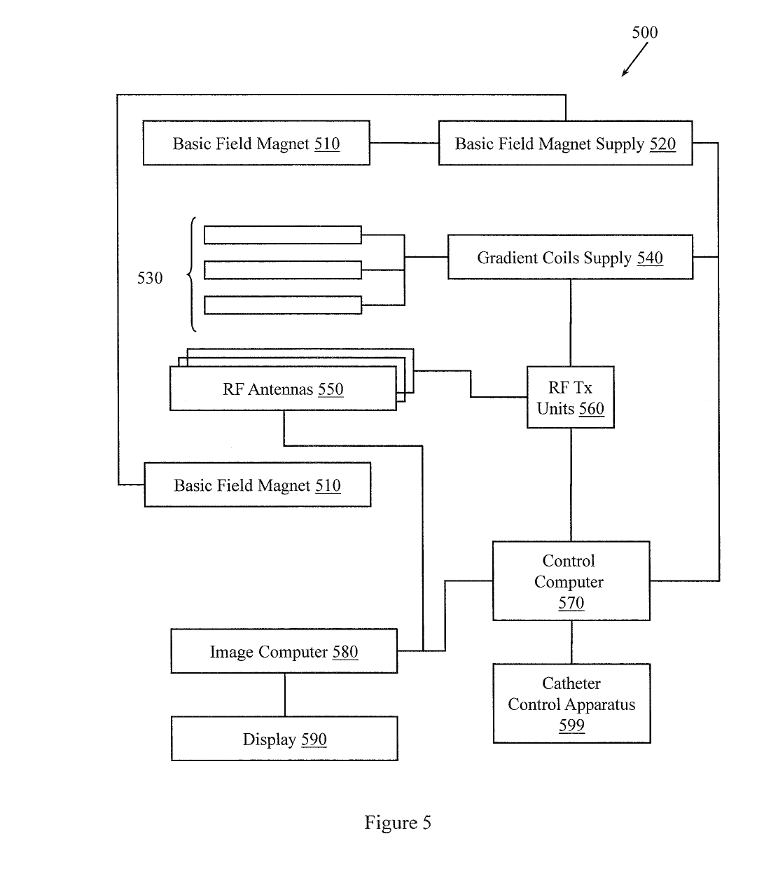

FIG. 5 illustrates an example MRI apparatus 500 configured with a catheter control apparatus 599 that is configured to facilitate IMRI guided catheter control. The catheter control apparatus 599 may be configured with elements of example apparatus described herein and/or may perform portions of example methods described herein. Thus, MRI apparatus 500 and catheter control apparatus 599 may provide means (e.g., hardware, software, firmware) for reshaping a catheter configured with a 3D array of steering coils by selectively providing current to at least one member of the 3D array of steering coils. The MRI apparatus 500 may also provide means (e.g., hardware, software, firmware) for acquiring a first image of a region associated with the catheter while current is provided to at least one member of the 3D array of steering coils. The MRI apparatus 500 may also provide means (e.g., hardware, software, firmware) for acquiring a second image of a region associated with the catheter while no current is provided to any member of the 3D array of steering coils. Since two images are available, one of the vasculature near the catheter and one that includes a signal void that identifies the location of the catheter, MRI apparatus 500 may also provide means (e.g., hardware, software, firmware) for passively tracking the catheter based, at least in part, on one or more of, the first image, and the second image. For example, a representation of the catheter may be superimposed on an image based on the location of the signal void.

The apparatus 500 includes a basic field magnet(s) 510 and a basic field magnet supply 520. Ideally, the basic field magnets 510 would produce a uniform B₀ field. However, in practice, the B₀ field may not be uniform, and may vary over an object being imaged by the MRI apparatus 500. MRI apparatus 500 may include gradient coils 530 configured to emit gradient magnetic fields like G_{S}, G_{P} and G_{R}. The gradient coils 530 may be controlled, at least in part, by a gradient coils supply 540. In some examples, the timing, strength, and orientation of the gradient magnetic fields may be controlled, and thus selectively adapted during an MRI procedure.

MRI apparatus 500 may include a set of RF antennas 550 that are configured to generate RF pulses and to receive resulting magnetic resonance signals from an object to which the RF pulses are directed. In some examples, how the pulses are generated and how the resulting MR signals are received may be controlled and thus may be selectively adapted during an MRI procedure. Separate RF transmission and reception coils can be employed. The RF antennas 550 may be controlled, at least in part, by a set of RF transmission units 560. An RF transmission unit 560 may provide a signal to an RF antenna 550.

The gradient coils supply 540 and the RF transmission units 560 may be controlled, at least in part, by a control computer 570. In one example, the control computer 570 may be programmed to control an IMRI device as described herein. The magnetic resonance signals received from the RF antennas 550 can be employed to generate an image and thus may be subject to a transformation process like a two dimensional FFT that generates pixilated image data. The transformation can be performed by an image computer 580 or other similar processing device. The image data may then be shown on a display 590. While FIG. 5 illustrates an example MRI apparatus 500 that includes various components connected in various ways, it is to be appreciated that other MRI apparatus may include other components connected in other ways.

FIG. 9 illustrates forces associated with single coil catheter tip deflection as described by University of Toronto Student F Settecase in the Master's Thesis titled: Magnetically-Assisted Remote Control (MARC) Steering of Endovascular Catheters for Interventional MRI: A Model For Deflection and Design Implications, as reported in Med Phys. 2007 August; 34(8):3135-42. This work cites the previous single coil catheter deflection work performed by Roberts, et al.

Settecase's thesis described an equation that characterizes the relationship between deflection and a number of physical factors in single coil catheters. Deflection was described according to Settecase's equation:

θ/sin(γ−θ)=nIABL/EIA,

where θ is the deflection angle, n is the number of solenoidal turns, I is the current, A is the cross-sectional area of the catheter tip, B is the MR scanner main magnetic field, L is the unconstrained catheter length, E is Young's Modulus for the catheter material, IA is the area moment of inertia, and y is the initial angle between the catheter tin and magnetic field B.

Note that deflection depends on the initial angle γ between the catheter tip and the main magnetic field. The catheter tip is deflected by a magnetic torque that is a function of applying current to the coil in the catheter. The magnetic torque is the cross product of the magnetic moment m and field B. The magnetic torque is produced because current running through a wire solenoid induces a magnetic moment in the solenoid. The magnetic moment is characterized by:

m=μnIA

where μ is the magnetic permeability of a liquid in which the solenoid is placed, n, is the number of solenoid turns, I is the current applied, and A is the cross-sectional area of the solenoid. Recall that a magnetic scanner produces a substantially homogenous and substantially constant magnetic field. Therefore, a magnetic moment created in a solenoid will experience a torque described by:

τmag=m×B.

This torque may also be described by:

τmag=mB sin(γ−θ),

where m is the magnetic moment vector and θ is the angle through which the catheter tip is deflected. γ−θ is the final angle between m and B. The magnetic torque causes the device housing the solenoid (e.g., catheter tip) to move relative to the main magnetic field until γ=θ. How far the device housing the solenoid will move depends on factors including the stiffness of the device, the current applied, the current polarity, and so on.

Example systems and methods include multiple elements that can be individually controlled to facilitate reshaping a device located in a constant magnetic field. The individual elements may be coils, the device may be a catheter, and the constant magnetic field may be produced by an MRI scanner.

While example systems, methods, and so on have been illustrated by describing examples, and while the examples have been described in considerable detail, it is not the intention of the applicants to restrict or in any way limit the scope of the appended claims to such detail. It is, of course, not possible to describe every conceivable combination of components or methodologies for purposes of describing the systems, methods, and so on described herein. Therefore, the invention is not limited to the specific details, the representative apparatus, and illustrative examples shown and described. Thus, this application is intended to embrace alterations, modifications, and variations that fall within the scope of the appended claims.

To the extent that the term “includes” or “including” is employed in the detailed description or the claims, it is intended to be inclusive in a manner similar to the term “comprising” as that term is interpreted when employed as a transitional word in a claim.

To the extent that the term “or” is employed in the detailed description or claims (e.g., A or B) it is intended to mean “A or B or both”. When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995).

To the extent that the phrase “one or more of, A, B, and C” is employed herein, (e.g., a data store configured to store one or more of, A, B, and C) it is intended to convey the set of possibilities A, B, C, AB, AC, BC, and/or ABC (e.g., the data store may store only A, only B, only C, A&B, A&C, B&C, and/or A&B&C). It is not intended to require one of A, one of B, and one of C. When the applicants intend to indicate “at least one of A, at least one of B, and at least one of C”, then the phrasing “at least one of A, at least one of B, and at least one of C” will be employed.

What is claimed is:

1. A computer-implemented method, comprising:
determining one or more of, a current amount, and a current polarity, for a current to be provided to at least one member of a three dimensional (3D) array of steering coils coupled to a catheter positioned in a magnetic field produced by an MRI apparatus,
where the current amount and the current polarity are configured to produce a magnetic torque sufficient to bend the catheter in a desired direction and in a desired amount, where the magnetic torque is a result of a magnetic moment induced in the at least one member of the 3D array of steering coils as a function of an interaction between the magnetic field produced by the MRI apparatus and the total magnetic moment; and
controlling a current provider to provide the current having the current amount and current polarity to the at least one member of the 3D array of steering coils.

2. The computer-implemented method of claim 1, where the current amount and the current polarity are determined as a function of a desired navigational movement of the catheter during an endovascular procedure.

3. The computer-implemented method of claim 1, comprising:
controlling the MRI apparatus to acquire a first image in the region of the catheter while the current provider is providing current to at least one member of the 3D array of steering coils.

4. The computer-implemented method of claim 3, comprising:
controlling the MRI apparatus to acquire a second image in the region of the catheter while the current provider is not providing current to any member of the 3D array of steering coils; and
passively tracking the catheter based, at least in part, on the first image and the second image.

5. The computer-implemented method of claim 4, where controlling the MRI apparatus comprises selectively controlling the MRI apparatus to produce a pulse sequence that does not include slice select rewinding gradients.

6. The computer-implemented method of claim 1, comprising:
measuring a set of deflection angles associated with an inclination angle of the catheter with respect to the magnetic field produced by the MRI apparatus;
identifying a selected member of the 3D array of steering coils to be provided with a current based, at least in part, on a subset of the set of deflection angles; and
identifying the current amount and the current polarity for the current to be provided to the selected member of a 3D array of steering coils based, at least in part, on a subset of the set of deflection angles.

7. An apparatus, comprising:
a flexible housing configured to be bendable in three or more directions when located in a magnetic field;
a three dimensional array of elements coupled to the flexible housing, where members of the three dimensional array of elements are configured to be controlled individually with respect to current applied to the members, and where members of the three dimensional array of elements are configured to produce a magnetic torque sufficient to bend the flexible housing when supplied with a current while the apparatus is positioned in the magnetic field.

8. The apparatus of claim 7, the flexible housing being a catheter configured for use in an endovascular procedure, the magnetic field being generated by an MRI apparatus configured to acquire an image of the catheter during the endovascular procedure.

9. The apparatus of claim 7, the three dimensional array of elements being a three dimensional array of coils.

10. The apparatus of claim 8, the three dimensional array of elements being a three dimensional array of coils that includes at least two axial coils.

11. The apparatus of claim 10, where the three dimensional array of elements includes at least one side coil.

12. The apparatus of claim 11, where the at least one side coil is a square side coil.

13. The apparatus of claim 9, where the three dimensional array of elements includes two side coils and an axial coil.

14. The apparatus of claim 7, comprising:
a fiber optic element coupled to the housing, the fiber optic element being configured to provide a fiber optic image of an item in the region of the housing.

15. The apparatus of claim 7, comprising:
a laser ablation element coupled to the housing, the laser ablation element being configured to ablate an item in the region of the housing.

16. The apparatus of claim 7, comprising:
a current source coupled to the housing and to the three dimensional array of elements, the current source being configured to provide current to members of the three dimensional array of elements.

17. The apparatus of claim 16, the current source being a conductor configured to be connected to an external current provider.

18. The apparatus of claim 16, the current source being configured to have a current induced in the current source as the result of being exposed to radio-frequency (RF) energy.

19. A magnetic resonance imaging (MRI) apparatus, comprising:
means for reshaping a catheter configured with a 3D array of steering coils by selectively providing current to at least one member of the 3D array of steering coils.

20. The MRI apparatus of claim 19, comprising:
means for acquiring a first image of a region associated with the catheter while current is provided to at least one member of the 3D array of steering coils; and
means for acquiring a second image of a region associated with the catheter while no current is provided to any member of the 3D array of steering coils.

21. The MRI apparatus of claim 20, comprising:
means for passively tracking the catheter based, at least in part, on one or more of, the first image, and the second image.

22. A catheter, comprising:
an elongated body; and
a set of coils coupled to the elongated body on one side of a joint, the set of coils being configured to respond to a magnetic field by producing a relative motion of the elongated body about the joint.
